(19) United States
(12) Patent Application Publication
(10) Pub. No.: US 2022/0280384 A1
(43) Pub. Date: Sep. 8, 2022

(54) Thermosensitive Therapeutic Material Delivery Apparatus and Method of Use

(71) Applicant: FocalCool, LLC, Sewell, NJ (US)

(72) Inventors: Jennifer E. Mitchell, Elmer, NJ (US); Denise Merrill, Sewell, NJ (US); Thomas L. Merrill, Sewell, NJ (US)

(73) Assignee: FocalCool, LLC, Sewell, NJ (US)

(21) Appl. No.: 17/641,143

(22) PCT Filed: Nov. 6, 2020
(86) PCT No.: PCT/US20/59405

§ 371 Date: Mar. 8, 2022

Related U.S. Application Data
(60) Provisional application No. 62/931,660, filed on Nov. 6, 2019.

(51) Int. Cl. A61J 1/16; A61M 5/31; A61M 5/34; A61J 1/20

(52) U.S. Cl. CPC A61J 1/165; A61M 5/3134; A61M 5/347; A61J 1/201; A61M 2005/3103

(57) ABSTRACT

An apparatus and its method of use for the treatment of patients by the delivery of a temperature-controlled material. The apparatus and its method of use can be performed in conjunction with endoscopic ultrasound interventional procedures such as tumor imaging or biopsy. The apparatus can be integrated with the working channel of endoscopic ultrasound devices or other imaging devices (bronchoscope, colonoscope) or can operate as a stand-alone apparatus.

### CROSS-REFERENCE TO RELATED APPLICATIONS

This application claims the benefit of U.S. Provisional Patent Application Ser. No. 62/931,660, filed on Nov. 6, 2019, which is incorporated herein by reference in its entirety.

### BACKGROUND OF THE INVENTION

### Field of the Invention

The invention relates to an apparatus and its method of use for the delivery of a thermosensitive material into a patient.

### Description of the Related Art

The human body has a thermoregulatory system that maintains temperature precisely, variations of only +0.1° C. at 37° C. (normal body temperature) are common. The regulation represents a continuous balance between rest, work, heat, and thermal energy. Under some conditions, materials used to help the body also benefit by careful thermoregulation. One example (of many) is thermosensitive hydrogels (thermogels) that are liquids well below body temperature but solidify or form gels at or above body temperature. These thermogels are called reverse-phase polymers, meaning that these thermogels can revert to liquid phase below body temperature. Delivering, pumping, and distributing these thermogels in the body is difficult due to this phase change behavior and the inherent thermoregulatory system of the human body at warmer temperatures. As these thermogels warm, they become more viscous and less easily pumped or distributed in the body.

It would be beneficial to be able to insert therapeutic materials with properties that could be enhanced through thermal control into tissue.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

In one embodiment, the present invention is a material delivery system comprising a temperature-controlled reservoir that will maintain the material at a desired temperature prior to delivery, a coolant flow and temperature control system, a multi-lumen catheter that can act as a temperature-controlled sleeve for material delivery, and a material flow control system.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a side elevational view of an exemplary embodiment of an apparatus for the delivery of a thermosensitive material into a patient, inserted into an endoscopic ultrasound system (EUS);

FIG. 2 is a side elevational view of the apparatus of FIG. 1;

FIG. 3 is a perspective view of a syringe used to deliver a thermogel into the apparatus of FIG. 2;

FIG. 4 is an exploded view of the syringe of FIG. 3;

FIG. 5 is a sectional view of the syringe of FIG. 3;

FIG. 6 is a side elevational view of a sleeve assembly used with the apparatus of FIG. 2;

FIG. 7 is a lower perspective view of the sleeve assembly of FIG. 6;

FIG. 8 is a sectional view of the sleeve assembly of FIG. 6;

FIG. 9 is a sectional view of a sleeve of the sleeve assembly of FIG. 6, taken along lines 9-9 of FIG. 6;

FIG. 10 is a sectional view of a sleeve of the sleeve assembly of FIG. 6, taken along lines 10-10 of FIG. 6;

FIG. 11 is a side elevational view of an alternative exemplary embodiment of an apparatus for the delivery of a thermosensitive material into a patient, inserted into an EUS;

FIG. 12 is a side elevational view of the apparatus of FIG. 11;

FIG. 13 is a side elevational view of a sleeve assembly used with the apparatus of FIG. 12;

FIG. 14 is a lower perspective view of the sleeve assembly of FIG. 13;

FIG. 15 is a sectional view of the sleeve assembly of FIG. 13;

FIG. 16 is an exploded perspective view, in section of a distal tip of the sleeve of the sleeve assembly of FIG. 13;

FIG. 17 is a schematic view of the apparatus of either FIG. 1 or FIG. 11 inserted through a patient's stomach to access the patient's pancreas with a needle of the apparatus of either FIG. 1 or FIG. 11;

FIG. 18 is a graph of modulus and viscosity vs. temperature for a thermogel used with the apparatus of FIG. 1 or FIG. 11;

FIG. 19 is a graph showing needle length vs. temperature for the distal needle tip inside a patient; and

FIG. 20 is a graph showing rate of delivery of thermogel through the apparatus of either FIG. 1 or FIG. 11 vs. generated delivery pressures.

### DETAILED DESCRIPTION

In the drawings, like numerals indicate like elements throughout. Certain terminology is used herein for convenience only and is not to be taken as a limitation on the present invention. The terminology includes the words specifically mentioned, derivatives thereof and words of similar import. The embodiments illustrated below are not intended to be exhaustive or to limit the invention to the precise form disclosed. These embodiments are chosen and described to best explain the principle of the invention and its application and practical use and to enable others skilled in the art to best utilize the invention.

Reference herein to “one embodiment” or “an embodiment” means that a particular feature, structure, or characteristic described in connection with the embodiment can be included in at least one embodiment of the invention. The appearances of the phrase “in one embodiment” in various places in the specification are not necessarily all referring to the same embodiment, nor are separate or alternative embodiments necessarily mutually exclusive of other embodiments. The same applies to the term “implementation.”

As used in this application, the word “exemplary” is used herein to mean serving as an example, instance, or illustration. Any aspect or design described herein as “exemplary” is not necessarily to be construed as preferred or advantageous over other aspects or designs. Rather, use of the word exemplary is intended to present concepts in a concrete fashion.

The word “about” is used herein to include a value of +/−10 percent of the numerical value modified by the word “about” and the word “generally” is used herein to mean “without regard to particulars or exceptions.”

Additionally, the term “or” is intended to mean an inclusive “or” rather than an exclusive “or”. That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. In addition, the articles “a” and “an” as used in this application and the appended claims should generally be construed to mean “one or more” unless specified otherwise or clear from context to be directed to a singular form.

Unless explicitly stated otherwise, each numerical value and range should be interpreted as being approximate as if the word “about” or “approximately” preceded the value of the value or range.

The use of figure numbers and/or figure reference labels in the claims is intended to identify one or more possible embodiments of the claimed subject matter in order to facilitate the interpretation of the claims. Such use is not to be construed as necessarily limiting the scope of those claims to the embodiments shown in the corresponding figures.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention.

Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

The present invention is a thermosensitive therapeutic material delivery apparatus and system that can provide temperature-controlled material delivery directly into a body, such as a cancerous lesion or infarct region or organ or cyst, during an endoscopic ultrasound procedure or during other types of interventional procedures. Temperature control of the material allows an aliquot to be delivered in liquid form and transition to a gel only within the lesion, or region, or organ. The aliquot can be a thermogel (blank or including drugs), stem cells, embolizing agents, chemoembolozing agent, or other therapeutic material with physical properties that benefit from temperature control.

While an exemplary use for the inventive apparatus is to deliver a thermogel into an organ, those skilled in the art will recognize that the inventive apparatus can be used to deliver any type of aliquot into any region in a living body, whether it be human or animal.

In an exemplary embodiment, the apparatus integrates with the working channel of a device such as an endoscopic ultrasound system (EUS). Alternatively, the apparatus can be a stand-alone device as well and can be used without a separate supporting device. Standard catheterization devices and procedures would also be amenable to this apparatus and method of use.

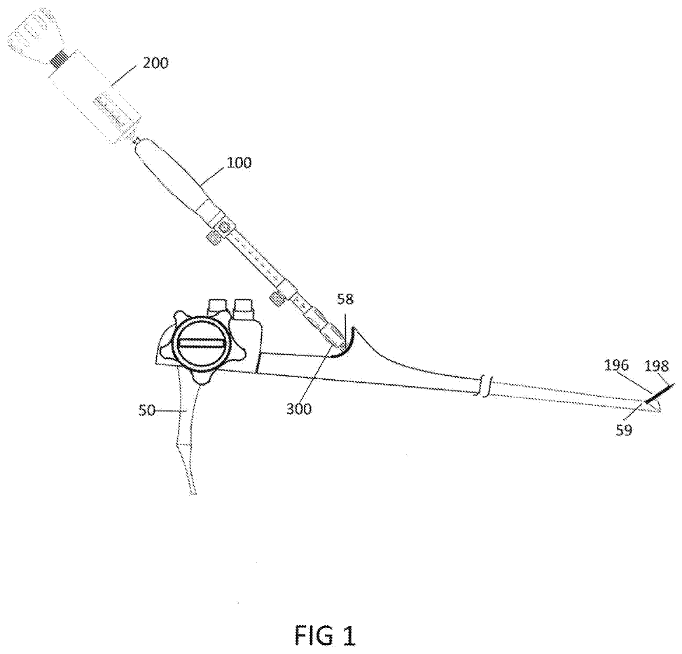

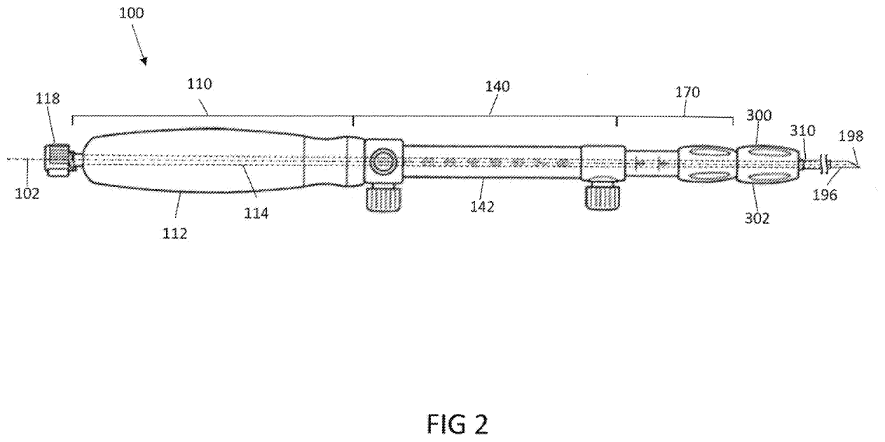

An exemplary thermosensitive therapeutic material delivery apparatus 100 (“apparatus 100”) according to the present invention is shown in a standalone configuration in FIG. 1 and incorporated into a known EUS 50 in FIG. 2.

Apparatus 100 includes a proximal portion 110, a mid portion 140 extending distal of proximal portion 110, and a distal portion 170 extending distally of mid portion 140. A needle 196 extends distally from proximal portion 110, through apparatus 100, and out of distal portion 170. Needle 196 is inserted into the targeted lesion to deliver the aliquot. Apparatus 100 includes a longitudinal axis 102 extending through proximal portion 110, mid portion 140, and distal portion 170.

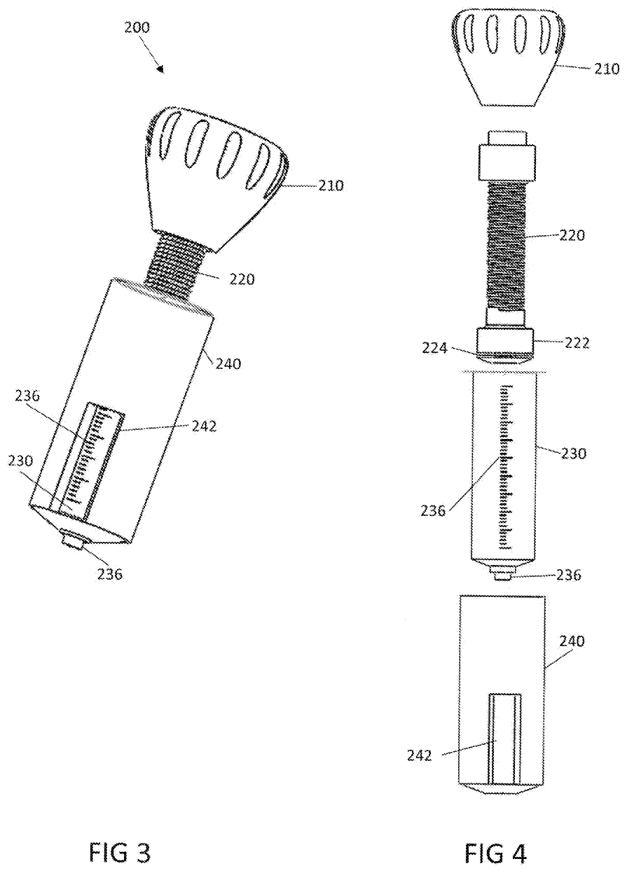

Proximal portion 110 includes a proximal handle 112 having an axial passage 114 extending therethrough. Axial passage 114 extends along longitudinal axis 102 through proximal portion 110, mid portion 140, and distal portion 170. Proximal portion 110 also includes a connection point 118 for a thermogel supply syringe 200, shown in FIGS. 3-5, that is to be injected through apparatus 100 to a target delivery location in the patient. A hollow needle 196 extends distally out of apparatus 100 from distal portion 170 to a distal needle tip 198. Syringe 200 can be stored in a chilled location, such as a refrigerator or freezer prior to use, and can be removed from the storage location and attached to apparatus 100 immediately prior to sue.

Syringe 200 includes a proximal handle 210 connected to a threaded screw 220. Screw 220 is inserted into a container 230 that contains an aliquot of thermogel to be injected. Screw 220 mates with a corresponding internal screw 232 inside a proximal end 234 of container 230. A plunger 222 is disposed at a distal end 224 of screw 220. Screw 220 is rotated to advance plunger 222 so that the thermogel inside container 230 can be controllably injected into apparatus 100.

Container 230 includes a distal tip 236 that is removably connectable to connection point 118 on proximal handle 112 of apparatus 100. An exterior of container 230 can include graduation indicia 236 to provide an indication to a clinician regarding how much of the aliquot is in container 230 during the injection of the aliquot. Additionally,

A thermal sleeve 240 surrounds container 230 and thermally insulates container 230 to reduce heat transfer from the ambient environment to container 230, which would result in undesired heating of the aliquot in container 230. A window 242 in thermal sleeve 240 allows graduation indicia 236 to be visualized. Additionally, the background for graduation indicia 236 can be constructed from a thermosensitive color gradient material that changes colors according to temperature.

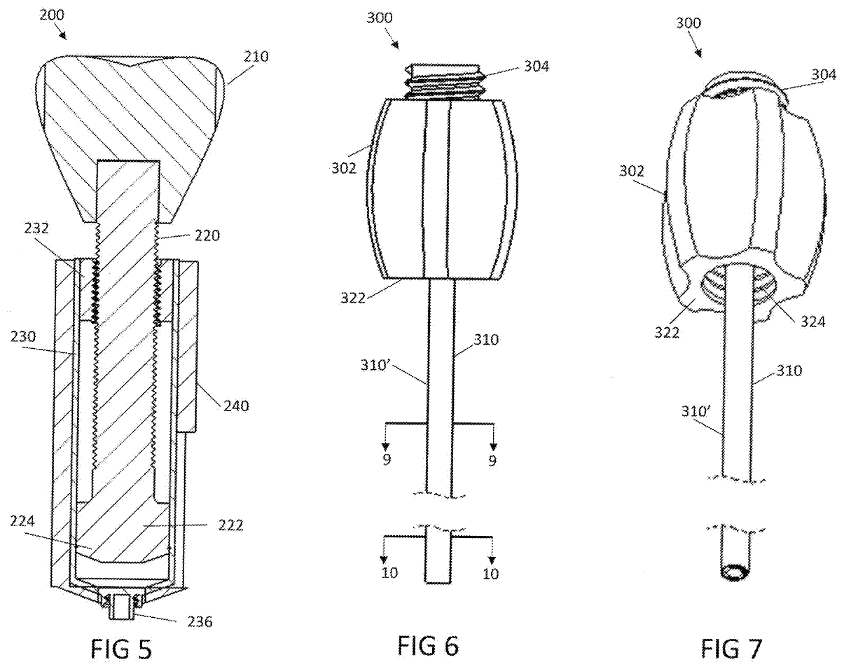

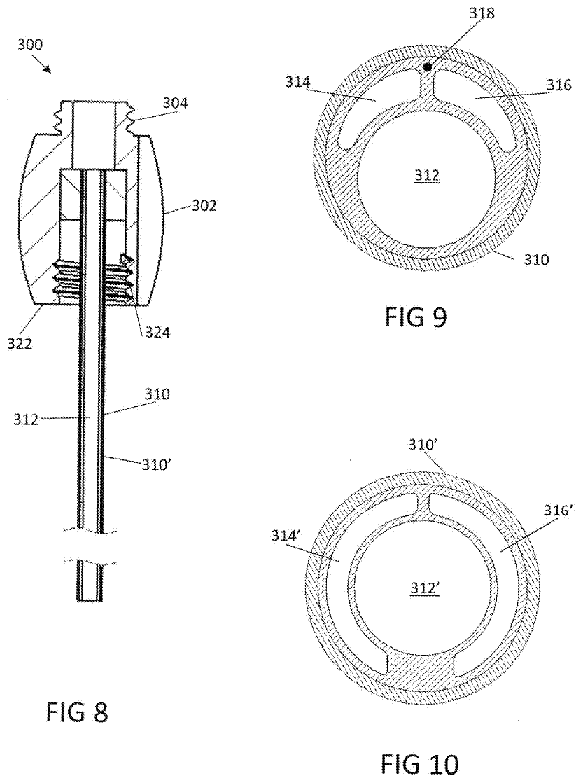

A sleeve assembly 300, shown in FIGS. 6-8, is used to reduce the amount of temperature increase of the aliquot as the aliquot is advanced through needle 196 from distal portion 170 to distal needle tip 198. Sleeve assembly 300 includes a hollow body 302 having a threaded proximal end 304 that can be threadingly engaged with distal portion 170. Body 302 includes a proximal lumen 308 that, when sleeve assembly 300 is attached to apparatus 100, is in fluid communication with axial passage 114. Proximal lumen 308 is in fluid communication with an insulating sleeve 310 that extends distally from body 302 to surround needle 196. Sleeve 310 includes an inner insulating lumen 312 that extends over a length of insulating sleeve 310.

In an exemplary embodiment, as shown in FIG. 9, insulating sleeve 310 comprises inner insulating lumen 312, a first insulating lumen 314, and a second insulating lumen 316. Optionally, a guide wire lumen 318 can also be provided.

Insulating sleeve 312 is inserted over needle 196 and first and second insulating lumens 314, 316 can include have a full or partial vacuum, be filled with air or an insulation, such as polyurethane or aerogel particles, or a phase change material such as a cold pack gel.

Alternatively, as shown in FIG. 10, an insulating sleeve 310′ can include a concentric lumen 312′ for aliquot delivery, surrounded by “C” shaped insulating lumens 314′, 316′ to store cooling fluid around lumen 312′.

Body 302 further includes a distal end 322 having a threaded portion 324. Threaded portion 324 is configured for removable threaded connection to a port 58 on EUS 50 (shown in FIG. 1). Threaded portion 324 is a female thread, whereas threaded proximal end 304 is a male thread so that, if a non-temperature dependent aliquot is being administered to the patient, sleeve assembly 300 can be omitted and distal portion 170 can be directly connected to port 58 on EUS 50.

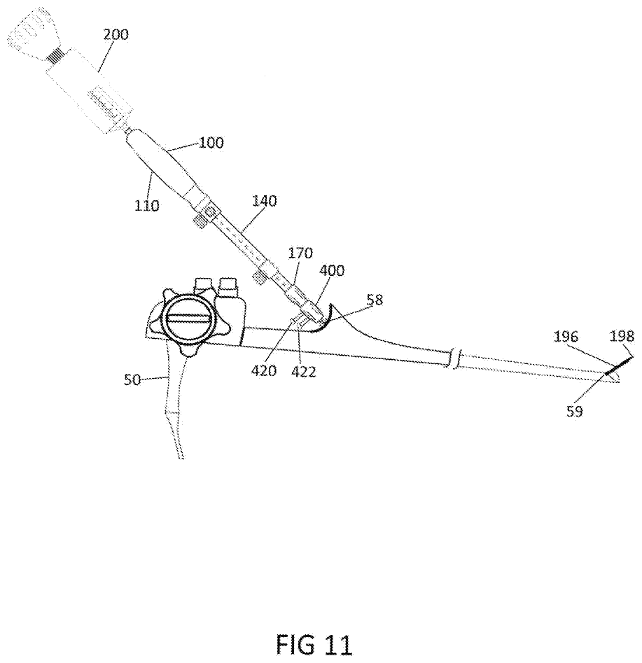

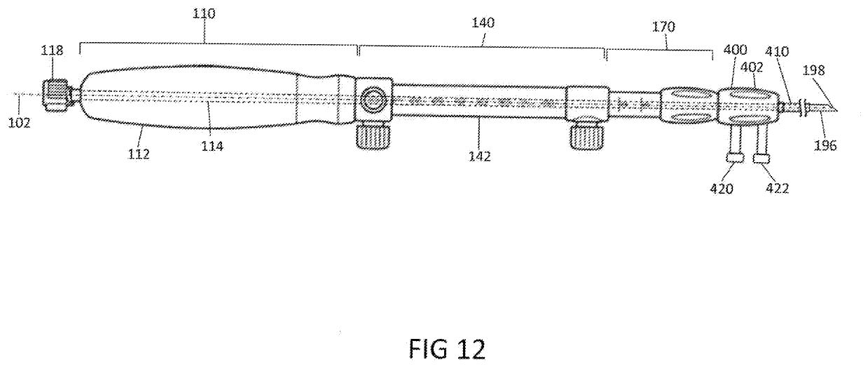

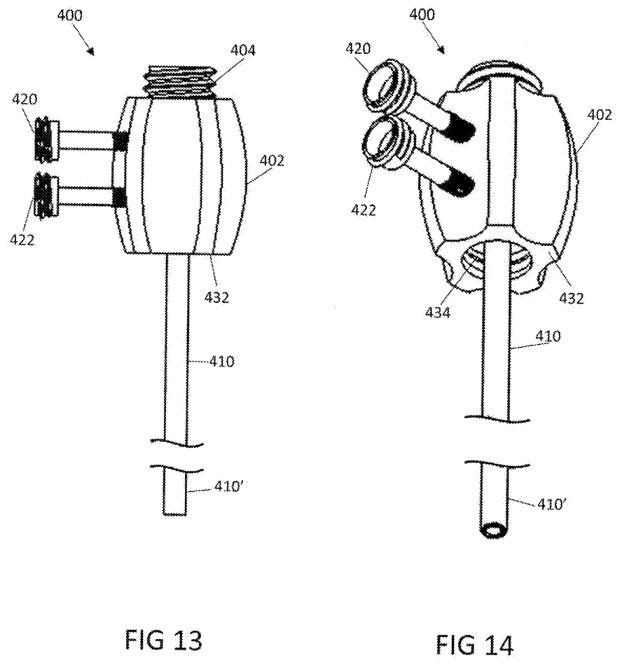

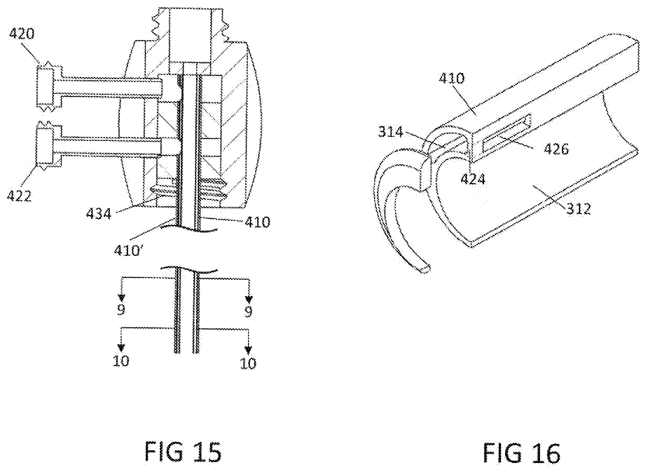

Referring to FIGS. 11-15, instead of a static cooling medium for needle 196 in sleeve assembly 300, a sleeve assembly 400 can provide active cooling of needle 196. Sleeve assembly 400 includes a hollow body 402 having a threaded proximal end 404 that can be threadingly engaged with distal portion 170. Body 402 includes a proximal lumen 408 that, when sleeve assembly 400 is attached to apparatus 100, is in fluid communication with distal portion 170. Proximal lumen 408 is in fluid communication with an insulating sleeve 410 that extends distally from body 402 to surround needle 196.

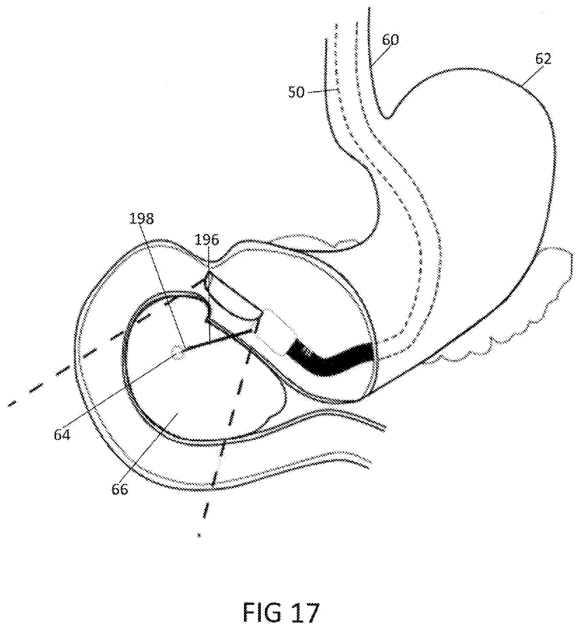

Sleeve assembly 400 further includes ports 420, 422 to transmit a cooling medium to sleeve 410. The cooling medium can be a cooled fluid, such as saline at about 4 degrees Celsius, to reduce ambient heat transfer to sleeve 410. Exemplary cross sections of sleeve 410, 410′ are shown in FIGS. 9 and 10 and are described above with respect to sleeve assembly 300. However, due to the flowing nature of the fluid in sleeve assembly 400, as shown in FIG. 17, a distal end 424 of sleeve 410 includes a cross-connect 426 that provides fluid communication between first insulating lumen 314 and second insulating lumen 316 (not shown).

Body 402 further includes a distal end 432 having a threaded portion 434. Threaded portion 434 is configured for removable threaded connection to a port 58 on EUS 50 (shown in FIG. 1).

Apparatus 100 can be used with syringe 200 and sleeve assembly 300 or sleeve assembly 400 to inject a thermogel or other aliquot into a targeted area, such as a lesion 64, in a patient, as shown in FIG. 17.

Following identification of the lesion 64 and assessment of desired amount and type of anti-cancer drug to be delivered, an appropriately sized aliquot (3 ml—50-75% of lesion volume) of thermogel with optimal kinetic properties (of both thermogel concentration and anti-cancer drug concentration) is removed from cold storage (for example, at 4° C.). Apparatus 100 is inserted into the auxiliary or working channel of the endoscope 50 via port 58 and connected via the threaded connection 324, 434. Fine needle injector handle adjustments may be needed to appropriately integrate with the EUS 50.

With apparatus 100 and EUS 50 connected, needle 196 is advanced into lesion 64 at the desired depth (4-8 cm into lesion). This will ensure accurate penetration of the needle 196 into the desired location within the lesion 64. At this time, the insulated aliquot of drug laden thermogel will be connected to the connection port 118 of the handle 112. If sleeve assembly 400 is used, coolant is circulated through sleeve 410 via ports 420, 422.

Apparatus 100 and EUS 50 can be inserted down the esophagus 60 of the patient, through the stomach 62, so that distal needle tip 198 can extend out of opening 59 at a distal end of EUS 50 for insertion into a lesion 64 in a pancreas 66. While insertion of needle 196 into lesion 64 of pancreas 66 is shown, those skilled in the art will recognize that needle 196 can be inserted into other organs or body parts to dispense the thermogel.

The thermogel can be stored as small aliquots and maintained at a desired temperature in a small cooler (not shown) for a predetermined period of time. In an exemplary embodiment, the temperature can be 10 degrees Celsius or cooler. Desired aliquots can be removed from the cooler at time of use or can be stored in pre-charged container 230 with cooling or high insulation ability (e.g. ice, Styrofoam like material, etc.). In an exemplary embodiment, the amount of the aliquot is typically small, about 10 ml or less. In addition, the amount of cooling required to deliver thermogel to tissue inside an EUS 50 is modest, approximately 10 Watts.

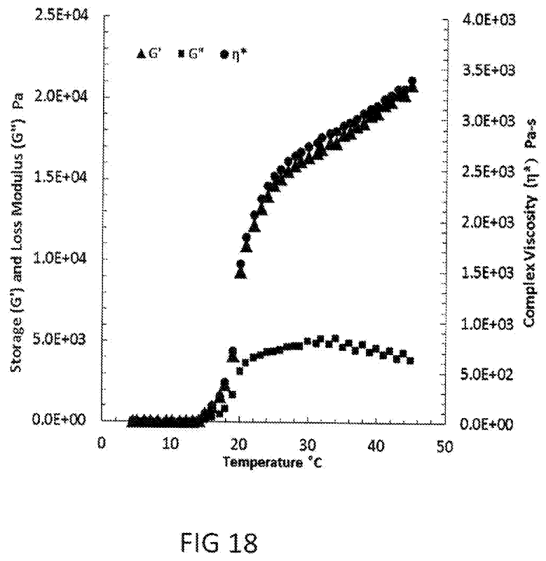

If the aliquot absorbs too much heat after removal from the cooler, the viscosity of the aliquot increases, making it more difficult for the aliquot to be advanced through apparatus 100 and into the target lesion. An exemplary graph showing increase in viscosity vs. an increase in temperature is shown in FIG. 18, with:

G′=Storage modulus (in Pa)

G″=Loss modulus (in Pa)

η*=Complex viscosity (Pa·s)

By way of example only, a 30% Pluronic mixture is a hydrogel with a liquid-to-gel transition temperature of 14° C. Viscosity values transition from <1 Pa-s at 4° C. to 3000 Pa-s at 37° C.

The storage modulus G′ is an indication of the hydrogel's ability to store deformation energy in an elastic manner. This is directly related to the extent of cross-linking; the higher the degree of cross-linking the greater the storage modulus. Swelling is also directly related to the degree of cross-linking, the more cross-linking the more swelling will be restricted.

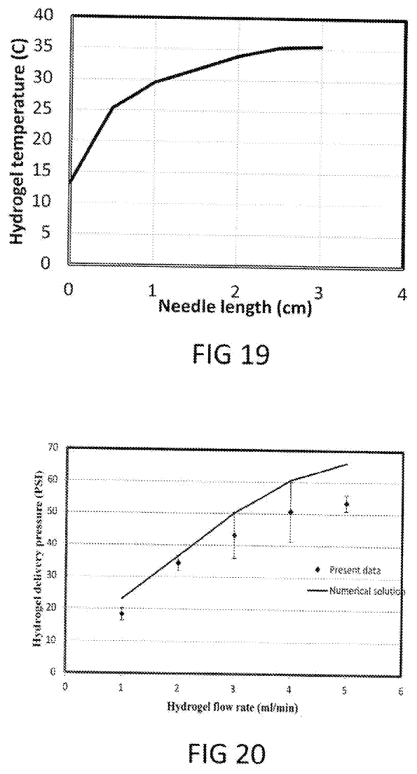

Further, distal needle tip 198 must extend distally from sleeve 310, 410 in order to be able to be inserted into the target organ, rending distal needle tip 198 uninsulated and subject to body heat, thereby risking unwanted heating of he thermogel at or around distal needle tip 198. FIG. 19 is a graph showing a length of needle 196 distal of sleeve 310, 410 and the associated temperature rise of that length of needle 196 within the patient. The graph shows that a temperature of distal needle tip 198 increases about 22 degrees Celsius over a length of 3 cm.

The significant temperature increase over the length of distal needle tip 198 encourages a quick administration of the thermogel to prevent congealment of the thermogel inside distal needle tip 198. FIG. 20, however, shows resulting delivery pressures associated with different delivery rates. Apparatus 100 must be constructed to withstand the pressures shown in the graph of FIG. 20 so that apparatus 100 does not burst or leak the thermogel material during delivery.

Once distal needle tip 198 is inserted into the target lesion 64, the thermogel can be injected into apparatus 100 from syringe 200 at connection point 118. The flow of thermogel into the lesion is controlled and varied as needed by advancing screw 220 distally toward distal tip 232. Once injected into apparatus 100, temperature is precisely controlled as the thermogel travels therethrough to prevent gel transition inside needle 196.

Temperature controlled thermogel travels through needle 196 to distal needle tip 198. In one embodiment, the temperature of the thermogel is kept cool as it travels through needle 196 by its close proximity to coolant in sleeve 310 or sleeve 410.

In an exemplary embodiment, thermogel can be injected at a rate of 0.5 ml/min and needle 196 is gradually retracted proximally during the injection to yield a solidified thermogel implant with partial infusion into the lesion 64 and partial location within the space previously occupied by needle 196. Prior to removal of needle 196 from the tissue 66, thermogel delivery is stopped as thermogel within the lesion 64 transitions to a solid. This will prevent leakage of the thermogel from lesion 64 before the thermogel solidifies. The insulated needle 196 prevents or retards solidification of thermogel within needle 196.

Once the implant is adequately solidified in the lesion 64, imaging can be used to qualify/quantify thermogel formation. If required or desired, injection and thermogel delivery can be repeated as prescribed. Once the anti-cancer drug encapsulated within the thermogel is implanted within the cancerous lesion 64, the thermogel will remain in situ and release the drug directly into the target organ 66 for several days or weeks.

The invention has the following advantages over the prior art, including precise temperature control from reservoir storage to delivery of material from outside the body to a location inside the body; precise flow control from reservoir storage to delivery of material from outside the body to a location inside the body.

Precise pressure control from reservoir storage to delivery of material from outside the body to a location inside the body; full integration with existing technology such as EUS, endobronchial ultrasound, and other standard interventional tools for minimal impact on care path complexity; and new opportunities for material development and delivery that offer improved patient health care outcomes, including but not limited to increased cancer cell toxicity for cancer drugs, immunotherapies, improved viability of stem cells, and improved material properties to prevent cancer lesion ejection of cancer fighting agents.

It will be further understood that various changes in the details, materials, and arrangements of the parts which have been described and illustrated in order to explain the nature of this invention may be made by those skilled in the art without departing from the scope of the invention as expressed in the following claims.

What we claim is:

1. A thermosensitive therapeutic material delivery apparatus comprising:
a proximal portion extending along a longitudinal axis;
a proximal connection extending proximally of the proximal portion;
a distal portion extending distal of the proximal portion, the distal portion extending along the longitudinal axis;
a distal connection extending distally of the distal portion; and
a needle extending distally outwardly from the distal portion.

2. The thermosensitive therapeutic material delivery apparatus according to claim 1, further comprising a syringe releasably connectable to the proximal connection.

3. The thermosensitive therapeutic material delivery apparatus according to claim 2, wherein the syringe comprises a container and a thermal sleeve surrounding the container.

4. The thermosensitive therapeutic material delivery apparatus according to claim 3, wherein the syringe comprises a screw and wherein the container comprises a mating screw thread.

5. The thermosensitive therapeutic material delivery apparatus according to claim 1, further comprising a sleeve assembly releasably connectable to the distal connection.

6. The thermosensitive therapeutic material delivery apparatus according to claim 5, wherein the sleeve assembly comprises a distal connection configured to releasably connect to an insertable device.

7. The thermosensitive therapeutic material delivery apparatus according to claim 6, wherein the insertable device comprises an endoscopic ultrasound device.

8. The thermosensitive therapeutic material delivery apparatus according to claim 6, wherein the sleeve assembly comprises a lumen extending distally of the distal connection.

9. The thermosensitive therapeutic material delivery apparatus according to claim 8, wherein the lumen is configured to slide over the needle.

10. The thermosensitive therapeutic material delivery apparatus according to claim 8, wherein the lumen comprises an inner insulating lumen, a first insulating lumen, and a second insulating lumen.

11. The thermosensitive therapeutic material delivery apparatus according to claim 10, wherein the first insulating lumen is in fluid communication with the second insulating lumen.

12. The thermosensitive therapeutic material delivery apparatus according to claim 11, wherein the sleeve assembly comprises an inlet connection extending therefrom, the inlet connection being is in fluid communication with the first insulating lumen, and wherein the sleeve assembly comprises an outlet connection extending therefrom, the outlet connection being is in fluid communication with the second insulating lumen.

13. The thermosensitive therapeutic material delivery apparatus according to claim 10, wherein a phase change material is disposed in the first insulating lumen and the second insulating lumen.

14. The thermosensitive therapeutic material delivery apparatus according to claim 6, wherein the sleeve assembly further comprises a threaded proximal end.

15. The thermosensitive therapeutic material delivery apparatus according to claim 14, wherein the sleeve assembly distal connection comprises a female thread and the threaded proximal end comprises a male thread.
